(19) United States
(12) Patent Application Publication
(10) Pub. No.: US 2026/0242558 A1
(43) Pub. Date: Aug. 20, 2026

(54) HIGHLY TRANSPARENT THIN POLYVINYL ALCOHOL AEROGELS AND MANUFACTURE THEREOF

(71) Applicant: Northeastern University, Boston, MA (US)

(72) Inventors: Yi Zheng, Canton, MA (US); Xiaoli Li, Boston, MA (US)

(21) Appl. No.: 19/530,801

(22) Filed: Feb. 5, 2026

Related U.S. Application Data
(60) Provisional application No. 63/755,011, filed on Feb. 6, 2025.

(51) Int. Cl. C08J 9/28; A61L 15/42; B01D 39/16; E04F 13/18; E06B 7/00

(52) U.S. Cl. CPC C08J 9/283; A61L 15/425; B01D 39/1676; B01D 2239/10; B01D 2239/1216; B01D 2239/1241; B01D 2239/1291; C08J 2201/0502; C08J 2205/026; C08J 2205/042; C08J 2205/044; C08J 2205/048; C08J 2207/10; C08J 2329/04; E04F 13/18; E06B 7/00

(57) ABSTRACT

Disclosed are novel polyvinyl alcohol (PVA) aerogels created through an innovative one-step standing method.

### RELATED APPLICATIONS

This application claims the benefit of priority to U.S. Provisional Application No. 63/755,011, filed Feb. 6, 2025.

### GOVERNMENT SUPPORT

This invention was made with government support under Grant Number CBET-1941743 awarded by the National Science Foundation. The government has certain rights in the invention.

### BACKGROUND

Traditional aerogels, particularly silica aerogels, have been highly valued for their excellent thermal insulation and transparency. Their practical use is limited by their fragile, pearl-like internal structure, which makes them extremely brittle. This brittleness has been a significant drawback for broader applications, as many require stronger and more durable materials. Thus, there exists a need in the art to develop aerogels that exhibit strength and durability while also maintaining transparency of traditional aerogels.

### SUMMARY OF THE INVENTION

In certain aspects, the present disclosure provides an aerogel comprising polyvinyl alcohol (PVA);
wherein the aerogel comprises a plurality of small pores having an average small pore diameter; a plurality of large pores having an average large pore diameter; and
the average small pore diameter is less than the average large pore diameter.

In certain aspects, the present disclosure provides a process of preparing an aerogel comprising polyvinyl alcohol (PVA), comprising:
combining PVA and a first solvent, thereby producing a PVA solution;
introducing the PVA solution into a mold;
allowing the PVA solution to gel at ambient temperature for a gelling duration, thereby producing a PVA wet gel;
contacting the PVA wet gel with a second solvent to form a soaked PVA wet gel; and
drying the soaked PVA wet gel, thereby producing a PVA aerogel.

### BRIEF DESCRIPTION OF THE DRAWINGS

FIG. 1 shows a schematic diagram of the PVA aerogel fabrication process: (Panel a) wet gel prepared by freeze-thaw method; (Panel b) wet gel prepared by the one-step standing method described herein.

FIG. 2 shows Young's modulus of different aerogels, including PVA aerogel (described herein), silica aerogel, Cu NW aerogel, and graphene aerogel. The inset shows a transparent PVA aerogel produced through the one-step standing method.

FIG. 3 shows SEM images of the internal structure of PVA products. (Panels a,b) SEM images of the internal structure of PVA aerogels produced using the one-step standing method, corresponding to magnifications of 500× and 20,000×, respectively. (Panels c,d) SEM images of the internal structure of PVA films produced using the one-step standing method, corresponding to magnifications of 500× and 20,000×, respectively. (Panels e,f) SEM images of the internal structure of PVA aerogels produced using the freeze-thaw method, corresponding to magnifications of 500× and 20,000×, respectively. The bold-lined circles indicate the pores, and the short bold lines mark the diameter of the skeleton, which is considered the “particle size” in the text.

FIG. 4 contains two graphs showing (Panel a) the transmittance curves for PVA aerogels made using the one-step standing method and freeze-thaw method; and (Panel b) the linear attenuation coefficient curves for PVA aerogels made using the one-step standing method and freeze-thaw method.

FIG. 5 contains a series of graphs showing (Panel a) the curve of weight percentage versus time for one-step standing PVA aerogel, (Panel b) for pure PVA pellet, (Panel c) for PVA film; (Panel d) comparison curves of pure PVA pellet, PVA film, and one-step standing PVA aerogel at 100° C.; (Panel e) comparison curves and derivative curves of pure PVA pellet, PVA film, and one-step standing PVA aerogel at 400° C.; (Panel f) PVA aerogel residue after heating under 100° C., 200° C., 300° C., and 400° C. (The arrows in panel (e) indicate the corresponding axis meanings for the curves).

FIG. 6 shows thermal images of one-step standing PVA aerogel on a 50° C. hot plate: (Panel a) hot plate without the sample; (Panel b) hot plate with the sample after 5 s; (Panel c) hot plate with the sample after 30 s; (Panel d) hot plate with the sample after 60 s; (Panel e) the sample after the heating test; and (Panel f) the curve of the temperature versus time of the selected area shown in panel (d).

FIG. 7 shows another schematic diagram showing the aerogel preparation route described herein, and 12% aerogels produced through different gelling methods. Panel (a) shows transparent aerogel produced through the one-step standing method; Panel (b) shows opaque aerogel produced through the freeze-thawing method; Panel (c) shows a phase diagram showing the path of three different drying methods.

FIG. 8 shows during the displacement process, the plot of the DMSO residual weight over time for 10-15% PVA wet gels.

FIG. 9 shows opaque aerogels of different weight percentages: (Panel a) 2%; (Panel b) 4%; (Panel c) 10%; (Panel d) 12%; (Panel e) 20%; (Panel f) 28%; transparent aerogels with bubbles of different weight percentages: (Panel g) 20%; (Panel h) 24%; (Panel i) 28%; transparent and uniform aerogels of different weight percentages: (Panel j) 10%; (Panel k) 13%; (Panel l) 15%.

FIG. 10 is a graph showing the tensile modulus for PVA Aerogels with different concentrations.

FIG. 11 shows SEM images of PVA aerogels at different magnifications and concentrations.

FIG. 12 graphs pore sizes and particle sizes of PVA aerogels with different concentrations ranging from 10 to 15%.

FIG. 13 contains graphs showing (Panel a) the transmittance curves for transparent PVA aerogels ranging from 10-15% and opaque PVA aerogel; (Panel b) the curves of linear attenuation coefficient versus the wavelength for PVA aerogels ranging from 10-15% and opaque PVA aerogel.

### DETAILED DESCRIPTION

Aerogels have a unique structure, which leads to many exciting properties and potential applications. The properties of an aerogel are influenced by the inherent properties of the aerogel precursor as well as by the manufacturing process. The impact of different manufacturing processes on the properties of aerogel products cannot be ignored. One classic aerogel preparation process can be divided into three stages. They are the sol formation, sol-gel transition, and gel-aerogel transition processes. The commonly used method in the sol-gel process is the freeze-thawing method. During the freezing time, adjacent cross-linkable parts have enough time to connect, while thawing gives the internal structure more opportunities to form new cross-linked bonds, which are then repeated. The cycles take time, and the cross-linking points formed in different cycles influence the uniformity of the internal structure. The gel-aerogel transition process can also be called the drying process. The drying methods can be classified into three types based on the drying environment: under atmospheric conditions, under freezing, and in a supercritical state. Each method has advantages and disadvantages, and the choice should be made based on the specific research goals.

The disclosed PVA aerogels are the result of a highly engineered process designed to achieve high transparency. A supercritical drying method was selected to ensure a better porous structure. After initially determining the production process, PVA aerogels ranging from 2 to 28% were made. Based on such a wide concentration range, the production process was further refined, and the concentration range that could ensure the production of more stable aerogels was determined. Thus, described herein is a one-step gelling method for polymer aerogel precursors. This method allows the existence of transparent PVA aerogel and reduces the time required for the gelation process compared to the freeze-thawing method. Additionally, analyzing the fundamental relationship between transmittance and microstructure of aerogels enables this property to become tunable depending on the matrix.

Accordingly, the present invention is based on the surprising discovery that aerogels produced using a one-step cross-linking method comprise superior mechanical properties while maintaining transparency.

In certain aspects, the present disclosure provides an aerogel comprising polyvinyl alcohol (PVA);
wherein the aerogel comprises a plurality of small pores having an average small pore diameter; a plurality of large pores having an average large pore diameter; and
the average small pore diameter is less than the average large pore diameter.

In certain embodiments, the average small pore diameter and average large pore diameter may be measured by scanning electron microscopy, as described in Example 1.

In certain embodiments, the aerogel has a Young's modulus greater than 0.33 MPa. In certain embodiments, the aerogel has a Young's modulus of at least about 1 MPa. In certain embodiments, the aerogel has a Young's modulus of at least about 2 MPa. In certain embodiments, the aerogel has a Young's modulus of at least about 3 MPa. In certain embodiments, the aerogel has a Young's modulus of at least about 4 MPa. In certain embodiments, the aerogel has a Young's modulus of at least about 5 MPa. In certain embodiments, the aerogel has a Young's modulus of at least about 6 MPa.

In certain embodiments, the aerogel has a Young's modulus of about 0.01 MPa to about 100 MPa. In certain embodiments, the aerogel has a Young's modulus of about 0.1 MPa to about 100 MPa. In certain embodiments, the aerogel has a Young's modulus of about 1 MPa to about 100 MPa. In certain embodiments, the aerogel has a Young's modulus of about 1 MPa to about 10 MPa. In certain embodiments, the aerogel has a Young's modulus of about 1 MPa. In certain embodiments, the aerogel has a Young's modulus of about 2 MPa. In certain embodiments, the aerogel has a Young's modulus of about 3 MPa. In certain embodiments, the aerogel has a Young's modulus of about 4 MPa. In certain embodiments, the aerogel has a Young's modulus of about 5 MPa. In certain embodiments, the aerogel has a Young's modulus of about 6 MPa. In certain embodiments, the aerogel has a Young's modulus of about 7 MPa. In certain embodiments, the aerogel has a Young's modulus of about 8 MPa. In certain embodiments, the aerogel has a Young's modulus of about 9 MPa. In certain embodiments, the aerogel has a Young's modulus of about 10 MPa.

In certain embodiments, the aerogel comprises a hierarchical porous structure. A hierarchical porous structure means a structure containing pores of different length scales (e.g., mesopores and macropores). Hierarchical porous structures may be characterized by their (1) multiple pore size scales, (2) interconnectivity of the pores, and (3) regularity.

In certain embodiments, the plurality of small pores and a plurality of large pores are substantially uniformly distributed in the aerogel.

In certain embodiments, the average small pore diameter is about 10 to about 100 nm.

In certain embodiments, the small pores are mesopores.

In certain embodiments, the small pores are holes. In certain embodiments, the small pores are indentations. In certain embodiments, the small pores are cavities. In certain embodiments, the small pores are concave recessions.

In certain embodiments, the average large pore diameter is about 100 nm to about 1000 nm.

In certain embodiments, the large pores are macropores.

In certain embodiments, the large pores are indentations. In certain such embodiments, the large pores are indentations and the small pores are holes within the indentations of the large pores. In other embodiments, the large pores are indentations and the small pores are smaller indentations within the indentations of the large pores.

In certain embodiments, the aerogel comprises large pores that define primary pore cavities. In certain such embodiments, smaller pores are formed within pore walls or skeletal framework surrounding the large pores, thereby establishing a hierarchical porous microstructure.

In certain embodiments, the aerogel comprises a plurality of particles having a particle size of about 10 to about 100 nm.

In certain embodiments, the aerogel exhibits Rayleigh scattering.

In certain embodiments, the aerogel has transmittance values of about 30% to about 85% in the visible light spectrum (380-700 nm).

In certain embodiments, the aerogel has transmittance values of about 10% to about 95% in the near-infrared light spectrum (800-2500 nm). In certain embodiments, the aerogel has transmittance values of about 90% to about 94% in the near-infrared light spectrum (800-2500 nm).

In certain embodiments, a portion of the small pores are positioned within the large pores.

In further aspects, the present disclosure provides a building material, comprising the aerogel described herein. In certain embodiments, the building material is selected from windows and facades.

In certain aspects, the present disclosure provides a medical device, comprising the aerogel of the present disclosure. In certain embodiments, the medical device is a dressing.

In certain aspects, the present disclosure provides aerospace equipment, comprising the aerogel of the present disclosure.

In certain aspects, the present disclosure provides a solar panel cover, comprising the aerogel of the present disclosure.

In certain aspects, the present disclosure provides a filter, comprising the aerogel of the present disclosure.

In certain aspects, the present disclosure provides a process of preparing an aerogel comprising polyvinyl alcohol (PVA), comprising:
combining PVA and a first solvent, thereby producing a PVA solution;
introducing the PVA solution into a mold;
allowing the PVA solution to gel at ambient temperature for a gelling duration, thereby producing a PVA wet gel;
contacting the PVA wet gel with a second solvent to form a soaked PVA wet gel; and drying the soaked PVA wet gel, thereby producing a PVA aerogel.

In certain embodiments, the PVA solution comprises about 10% to about 15% PVA by weight. In certain embodiments, the PVA solution comprises about 10% PVA by weight. In certain embodiments, the PVA solution comprises about 11% PVA by weight. In certain embodiments, the PVA solution comprises about 12% PVA by weight. In certain embodiments, the PVA solution comprises about 13% PVA by weight. In certain embodiments, the PVA solution comprises about 14% PVA by weight. In certain embodiments, the PVA solution comprises about 15% PVA by weight.

In certain embodiments, the first solvent is a polar aprotic solvent. In certain embodiments, the first solvent is DMSO.

In certain embodiments, the process further comprises heating the PVA solution before introducing the PVA solution to the mold.

In certain embodiments, the mold is a PTFE mold.

In certain embodiments, the gelling duration is about 12 hours to about 24 hours. In certain embodiments, the gelling duration is about 24 hours.

In certain embodiments, allowing the PVA solution to gel comprises cross-linking the PVA solution. In certain embodiments, cross-linking the PVA solution comprises forming hydrogen bonds between side-chain hydroxyl groups; that is, the hydroxyl groups that represent the side chains of the PVA units.

In certain embodiments, PVA is poorly soluble in the second solvent.

In certain embodiments, the second solvent is an alcohol. In certain embodiments, the second solvent is methanol.

In certain embodiments, contacting the PVA wet gel with a second solvent comprises soaking for about 1 day to about 4 days. In certain embodiments, contacting the PVA wet gel with a second solvent comprises soaking for about 1 day. In certain embodiments, contacting the PVA wet gel with a second solvent comprises soaking for about 2 days. In certain embodiments, contacting the PVA wet gel with a second solvent comprises soaking for about 3 days. In certain embodiments, contacting the PVA wet gel with a second solvent comprises soaking for about 4 days.

In certain embodiments, the process further comprises replacing the second solvent daily.

In certain embodiments, the soaked PVA wet gel comprises about 5% to about 0.1% by weight DMSO prior to drying. In certain embodiments, the soaked PVA gel comprises about 2% to about 0.1% by weight DMSO prior to drying. In certain embodiments, the soaked PVA gel comprises about 1% to about 0.1% by weight DMSO prior to drying.

In certain embodiments, the soaked PVA wet gel comprises about 5% by weight DMSO prior to drying. In certain embodiments, the soaked PVA wet gel comprises about 4% by weight DMSO prior to drying. In certain embodiments, the soaked PVA wet gel comprises about 3% by weight DMSO prior to drying. In certain embodiments, the soaked PVA wet gel comprises about 2% by weight DMSO prior to drying. In certain embodiments, the soaked PVA wet gel comprises about 1% by weight DMSO prior to drying. In certain embodiments, the soaked PVA wet gel comprises about 0.5% by weight DMSO prior to drying. In certain embodiments, the soaked PVA wet gel comprises about 0.1% by weight DMSO prior to drying.

In certain embodiments, the weight percent of DMSO may be measured using thermal gravimetric analysis, as described in Example 2.

In certain embodiments, drying the soaked PVA wet gel comprises ambient-pressure drying, freeze-drying, or supercritical carbon dioxide drying. In certain embodiments, drying the soaked PVA wet gel comprises supercritical carbon dioxide drying.

In certain aspects, the present disclosure provides an aerogel produced by the process described herein.

Disclosed herein are novel polyvinyl alcohol (PVA) aerogels created through an innovative one-step standing method. The one-step standing method revolutionizes the production of PVA aerogels by eliminating the limitations of the freeze-thaw method. It significantly shortens gelation time, removes the need for refrigeration, and produces aerogels with superior transparency. The resulting PVA aerogel features a hierarchical porous structure, with small pores within larger ones, promoting Rayleigh scattering that results in a light blue, transparent appearance, reaching up to 93.67% at a wavelength of 1333 nm.

Compared to existing aerogels, the PVA aerogels of the present disclosure have the following improvements and advantages:
1. The PVA aerogels produced using the one-step standing method exhibit significantly improved transparency (up to 93.67% at 1333 nm) and enhanced mechanical properties (Young's modulus of 6.18±0.56 MPa), addressing the balance issue between transparency and strength in traditional aerogels.
2. The one-step standing method reduces the gelation time compared to the freeze-thaw method, improving production efficiency and eliminating the need for refrigeration, which reduces energy consumption and environmental impact.
3. The simplified production process, combined with the reduced time and energy requirements, makes this PVA aerogel a more cost-effective solution.

The aerogels of the present disclosure are transparent, mechanically robust PVA aerogels. Such aerogels may be used in applications such as solar systems, where both optical clarity and material strength are crucial. The aerogels also have applications as energy-efficient building materials (e.g., building windows and facades), medical transparent dressings, aerospace equipment, as well as in green energy devices (such as solar panel covers).

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

As used herein, the term “aerogel” refers to a highly porous material of low density, typically prepared by forming a gel and then removing liquid from the gel while substantially retaining the gel structure.

As used herein, the term “uniform” can mean non-random, controlled to a predetermined range less than that which would result from uncontrolled conditions or random variation, and/or consistent through in at least one of structure, properties and characteristics.

As used herein, the term “Young's modulus” refers to a mechanical property of solid materials that measures the tensile or compressive stiffness when the force is applied lengthwise.

As used herein, the term “Rayleigh scattering” refers to the scattering or deflection of light, or other electromagnetic radiation, by particles with a size much smaller than the wavelength of the radiation. In certain embodiments, particle size may be measured by scanning electron microscopy, and the wavelength may be measured by spectrophotometry, as described in Example 1.

As used herein, the term “solvent” means any liquid substance capable of dissolving or dispersing one or more other substances. A solvent may be inorganic in nature, such as water, or it may be an organic liquid, such as ethanol, methanol, acetone, methyl acetate, ethyl acetate, hexane, petrol ether, dimethylsulfoxide (DMSO), etc.

As used herein, the term “mold” refers to a structure that is used as a tool to create a replicate part or product that has substantially the same size and shape as an original model part or parent mold.

As used herein, the term “cross-linking” refers to the formation of bonds between one polymer or portion of a polymer to another polymer or portion of a polymer.

As used herein, the term “pores” relates to regions of a porous article that are devoid of material. For example, a transverse area of porous body will comprise portions of the material forming the body and portions that are voids between the portions of material. Pores may be holes, indentations, cavities, concave recessions, or the like.

### EXAMPLES

The invention now being generally described, it will be more readily understood by reference to the following examples which are included merely for purposes of illustration of certain aspects and embodiments of the present invention and are not intended to limit the invention.

### Example 1: Synthesis and Characterization of Polyvinyl Alcohol Aerogels Using One-Step Standing Method

### Materials and Methods

### Materials

Dimethyl sulfoxide (CAS #67-68-5, lot #472301), purchased from Sigma-Aldrich Corporation (Burlington, MA, USA), was used as the solvent. The selected PVA is Mowiol 56-98 from Kuraray America Inc. (Chiyoda City, Japan), with a molecular weight of 105,600 g/mol, a degree of hydrolysis of 98.4±0.4 mol %, and a residual acetyl content of 1.5±0.4%. Methanol (CAS #67-56-1, lot #A43420) was purchased from the Thermo Fisher Scientific (Waltham, MA, USA).

### Methods

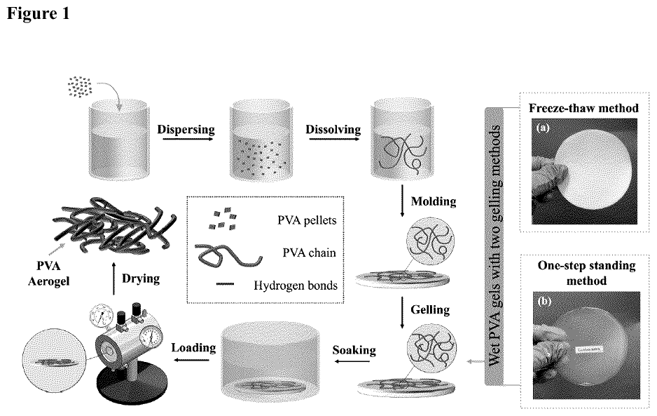

The PVA wet gel was prepared using the classic sol-gel method. The liquid inside the wet gel was then removed through supercritical drying, leaving only the PVA aerogel composed of a three-dimensional PVA skeleton. FIG. 1 shows a schematic diagram of the entire experimental process.

### Gelling Methods

The sol-gel method is a classic technique for preparing wet gels/hydrogels with the aim of transforming a solution into a gel. The side chains of PVA are hydroxyl groups, which can form hydrogen bonds when they come close to each other, resulting in physical cross-linking. Therefore, no additional chemical cross-linking agents were introduced in this process. Initially, PVA pellets were slowly added to the solvent DMSO, then heated and stirred to fully dissolve the PVA into the DMSO, forming a completely transparent PVA solution. The solution was then transferred into a mold for the subsequent sol-gel transition. The choice of mold can vary depending on the intended application. The mold selection underwent several iterations. Initially, a commercially purchased glass dish was used, but because the bottom of the dish was not flat enough, it led to uneven thickness in the final product. A chemically safe PTFE dish was then made, but due to the high viscosity of the polymer solution, the solution “crawled” up the walls of the dish, resulting in thicker edges and a thinner center in the final product. Finally, a custom-made PTFE plate was selected as the mold for forming thin sheet PVA aerogels, which allowed for the stable formation of thin aerogels with uniform thickness. The core idea of the sol-gel transition process is to use a gelling method to promote cross-linking within the solution. A commonly used method is the freeze-thaw method. As the name suggests, this method helps the solution cross-link through cycles of freezing and thawing.

After transferring the solution into the mold, both the mold and the solution were placed in a freezer for freezing. The freezing temperature was below the solvent's melting point to ensure that the movement of the solution is restricted. The purpose of freezing is to limit the movement of the polymer chains by freezing the solvent, giving the —OH groups that are already close to each other enough time to cross-link into hydrogen bonds. The sample was then thawed at room temperature to restore chain movement, providing additional opportunities for other-OH groups that had not yet cross-linked to come close and form hydrogen bonds. After several freeze-thaw cycles, the PVA chains in the solution cross-linked into a three-dimensional network structure. The freeze-thaw process involved freezing at 4° C. for 24 h, followed by thawing at room temperature for 12 h, repeating the cycle twice, and finally freezing again for 24 h before immersing the sample, along with the mold, in methanol, a poor solvent for PVA. Thus, the freeze-thaw process took 4 days to complete, and the resulting gel was opaque, as shown in FIG. 1, panel a. Described herein is a one-step standing method, which allows the PVA to cross-link while maintaining higher transparency, saving both time and energy, making it a more eco-friendly and sustainable approach. The specific procedure involves placing the mold and sample at room temperature for self-cross-linking for 24 h, followed by direct immersion in methanol. This significantly reduces the time required and results in transparent samples, as shown in FIG. 1, panel b. The purpose of soaking in methanol during this step is to replace the good solvent DMSO with a poor solvent for PVA, preventing the PVA from dissolving in DMSO. This allows the three-dimensional PVA network structure to be preserved, enabling the wet gel to proceed to the drying.

### Drying Methods

The drying process is essential for aerogels. The name “aerogel” comes from the fact that the drying process allows gas to replace the liquid in the wet gel. Common drying methods include ambient pressure drying, freeze-drying, and supercritical carbon dioxide drying. Each of these methods has advantages and disadvantages, and researchers typically choose based on the project's requirements. According to the classic phase diagram, these three methods can be distinguished. Ambient pressure drying directly transforms the substance from the liquid phase to the gas phase, crossing the liquid-gas boundary. However, the downside of crossing this boundary is the presence of surface tension, which can cause some collapse in the three-dimensional network structure. To avoid crossing the pressure boundary directly, freeze-drying and supercritical drying have emerged as alternatives. As the name suggests, freeze-drying involves freezing the substance from the liquid phase to the solid phase and then transitioning to the gas phase. This avoids crossing the liquid-gas boundary, but it still requires crossing the solid-gas boundary. During this transition, volume changes can occur, leading to some degree of structural collapse. Additionally, due to the freezing process, as in the freeze-thaw cycle of the sol-gel process, freeze-drying can result in a loss of transparency in PVA aerogels. Supercritical carbon dioxide drying, on the other hand, raises the temperature and pressure beyond the substance's critical temperature and pressure, reaching a supercritical phase, which is a mixture of liquid and gas. The transition to the gas phase from this supercritical phase avoids crossing any phase boundaries, thus preventing unwanted structural collapse. For this reason, supercritical carbon dioxide drying was chosen, as it also effectively preserves the transparency of the aerogel.

### Characterizations

### Scanning Electron Microscope (SEM)

A Zeiss Supra 25 scanning electron microscope, with an accelerating voltage of 5 kV, was used to image the internal structure of the aerogel. A Gatan high-resolution ion beam coater was used to coat all sample surfaces with a thin layer of conductive metal. After freezing with liquid nitrogen, the samples were fractured to obtain cross-sections, ensuring that the cross-sections remained intact without structural collapse caused by external forces.

### The Analysis for Recording Weight Loss During Heating

To assess the aerogels' thermal properties, thermogravimetric analysis was performed using a Q50 from TA Instruments (New Castle, DE, USA). This technique measures changes in sample mass as a function of temperature. This analysis was utilized to compare the thermal characteristics of PVA pellets, films, and aerogels.

### Dynamic Mechanical Analyzer (DMA)

Tensile tests were performed on PVA aerogels using a dynamic mechanical analyzer (2710-103 series, INSTRON Instrument, Norwood, MA, USA). The obtained Young's modulus was used to compare with that of other existing aerogels.

### Spectrophotometer (V-770)

The transparency of the aerogels was assessed using a JASCO V-770 UV-visible/NIR spectrophotometer (Easton, MD, USA), which covers a wavelength range of 300 nm to 2500 nm. The instrument operated with a scanning speed of 200 nm/min and a UV/VIS bandwidth of 5 nm.

### Results and Discussion

### Mechanical Property

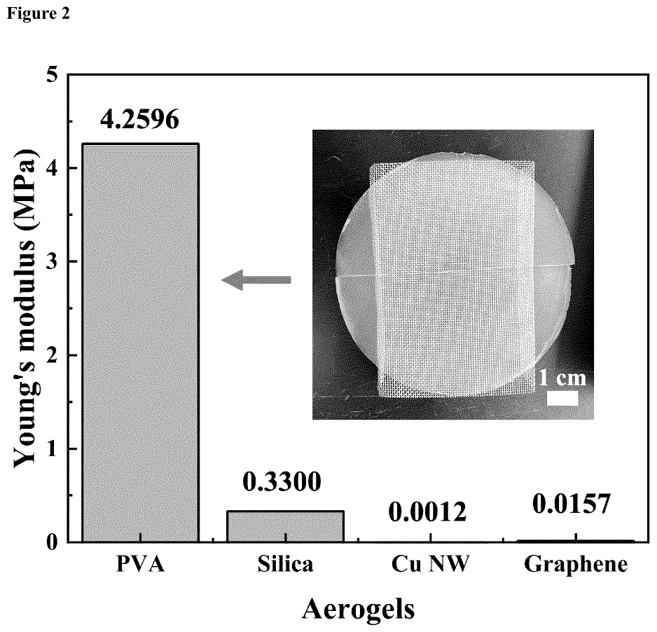

The focus of the processes and aerogels described herein included enhancing mechanical performance while maintaining transparency compared to silica aerogels. Therefore, tensile tests were conducted on the samples produced using the one-step standing method, and the obtained Young's modulus was compared with that of silica aerogels, Cu NW aerogels, and graphene aerogels from the literature. The samples were prepared by cutting long, thin strips from the PVA aerogel sheets using a new blade from top to bottom. The strips were approximately 10 mm in length, with the thickness corresponding to the thickness of the aerogel sheet, and the width was around 1-2 mm. Tensile tests were performed at room temperature using the 2710-103 series INSTRON Instrument. Cutting from top to bottom in one motion ensured that defects caused by the cutting process were avoided, ensuring accurate data. More than 10 valid tests were conducted, and the average value was taken to improve data accuracy. As shown in FIG. 2, the Young's modulus of the aerogels produced according to the processes described herein is significantly higher than the other three. Additionally, the inset shows that the PVA aerogel is transparent with a light blue tint. The one-step standing PVA aerogel demonstrates excellent flexibility due to the properties of the polymer material, allowing it to return to its original shape after bending. In contrast, the brittle silica aerogel breaks apart when pressure is applied with fingertips (data not shown). Additionally, the PVA aerogels were manually bent over 200 times, providing a more intuitive representation of the flexibility of PVA transparent aerogels (data not shown).

### Morphology

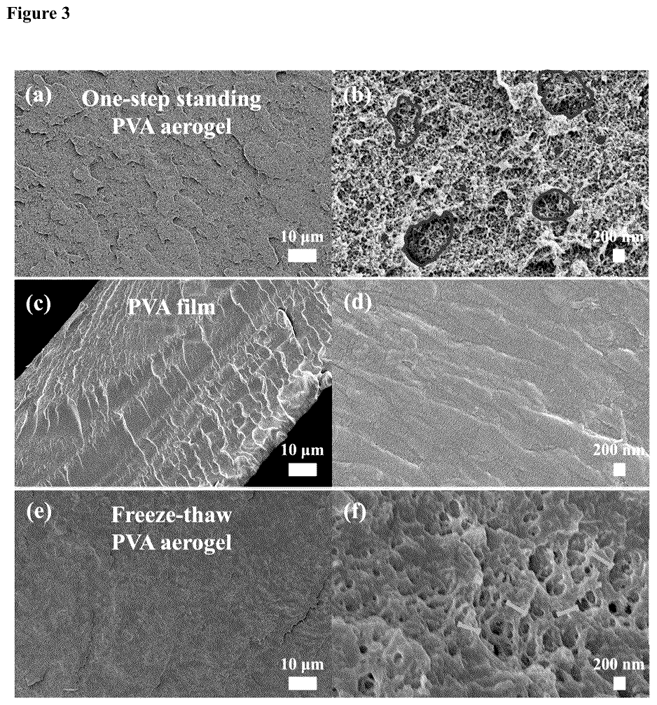

A scanning electron microscope was used to observe and image the PVA aerogels dried by supercritical carbon dioxide drying and the PVA film dried by ambient pressure drying. Different drying methods were applied to study how they affected the internal structure. To observe a clear and accurate representation of the internal structure, an intact cross-section is essential. However, common cutting methods, which apply downward pressure during slicing, were avoided as they could collapse the internal structure. Additionally, PVA products are flexible and difficult to fracture directly. Liquid nitrogen at a temperature as low as −195° C. was used to freeze the samples, making them brittle and allowing for clear fractures to obtain undamaged cross-sections. Theoretically, supercritical drying preserves the three-dimensional porous structure of the wet gel well, as mentioned earlier, because no phase boundary is crossed, avoiding volume changes and surface tension. As shown in FIG. 3, panel a, under 500× magnification, the internal structure of the PVA aerogel appears more uniform and porous. In contrast, the PVA film at the same magnification, as depicted in FIG. 3, panel c, looks very dense. Increasing the magnification to 20,000×, FIG. 3, panel b reveals an intriguing hierarchical porous structure within the aerogel. As highlighted by the red circles, numerous small pores exist within the larger pores. The small pores are measured in the tens of nanometers, while the larger pores are measured in the hundreds of nanometers. This hierarchical porous structure opens up various applications for PVA aerogels. For instance, the large pores could be used for drug delivery, providing more space for drug storage or adsorption, while the small pores help maintain the stability of the three-dimensional structure, making it less prone to collapse. In contrast, the internal structure of the PVA film shown in FIG. 3, panel d does not reveal any obvious porous structure. This reveals that the ambient pressure drying process nearly destroys all the porous structures of the PVA wet gels.

The internal structure of the polymer-based aerogels consists of a three-dimensional network made of polymer chains, so the particle size can be considered the width of the framework. FIG. 3, panels a-b shows the internal structure of transparent aerogels produced using the one-step standing method, while FIG. 3, panels e-f depicts the internal structure of opaque aerogels made using the freeze-thaw method. All four images reveal a porous structure, and a comparison of FIG. 3, panels b-f highlight a significant difference: the samples produced by the freeze-thaw method have a larger particle size, marked by the blue lines, on the order of hundreds of nanometers. In contrast, the particle size of the samples made using the one-step standing method is in the range of tens of nanometers.

From an optical perspective, scattering can be classified into two types: Mie scattering and Rayleigh scattering. Mie scattering occurs when the particle size is equal to or greater than the light wavelength, which explains why fog appears opaque and white. Rayleigh scattering, on the other hand, occurs when the particle size is much smaller than the light wavelength, which accounts for the blue color of the sky. Considering the particle sizes of the PVA aerogels, the samples produced by the freeze-thaw method are opaque, consistent with Mie scattering, while those made using the one-step standing method are transparent and appear light blue, aligning with Rayleigh scattering.

### Transmittance

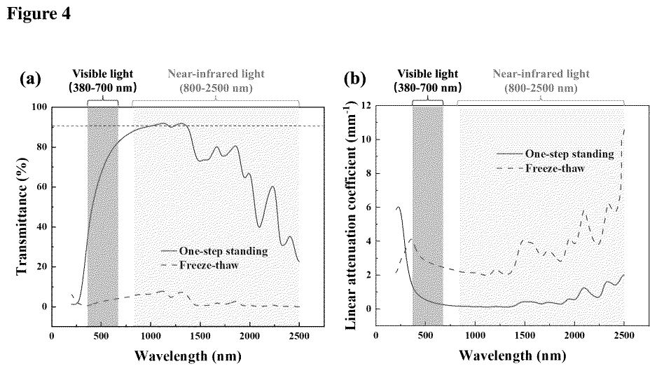

A V-770 UV-visible/NIR spectrophotometer was used to perform transmittance tests on aerogels produced by the one-step standing method and those produced by the freeze-thaw method. The wavelength range was set from 300 to 2500 nm, covering visible light and near-infrared light. The scanning speed was set at 200 nm/min. As shown in FIG. 4, panel a, the transmittance of the aerogels made using the one-step standing method is significantly higher than those made by the freeze-thaw method. However, transmittance is not only related to the material and internal structure but also to the thickness of the sample. Theoretically, any material with an infinitely small thickness would allow light to pass through. Therefore Beer-Lambert Law, as represented by Equation (1), was applied to eliminate the effect of thickness on transmittance [31]. In the equation, T represents the transmittance of the material [%], α is the linear attenuation coefficient [mm⁻¹], and x represents the sample thickness [mm]. The linear attenuation coefficient measures how challenging it is for physical entities like photons, sound waves, or electrons to travel through a material in one direction. For evaluating material transparency, a higher coefficient means that light diminishes quickly as it passes through, while a lower coefficient indicates easier light transmission. In other words, a smaller value reflects greater transparency.

$$T = {e}^{- α x} , \qquad ( 1 )$$

As shown in FIG. 4, panel b, calculations reveal that the one-step standing PVA aerogel still possesses superior light transmission compared to PVA aerogel made using the freeze-thaw method. This perfectly aligns with the earlier discussion about how particle size determines the scattering mechanism in a material. The one-step standing PVA aerogel follows Rayleigh scattering, resulting in better transparency and exhibiting a light blue color.

### Thermal Stability

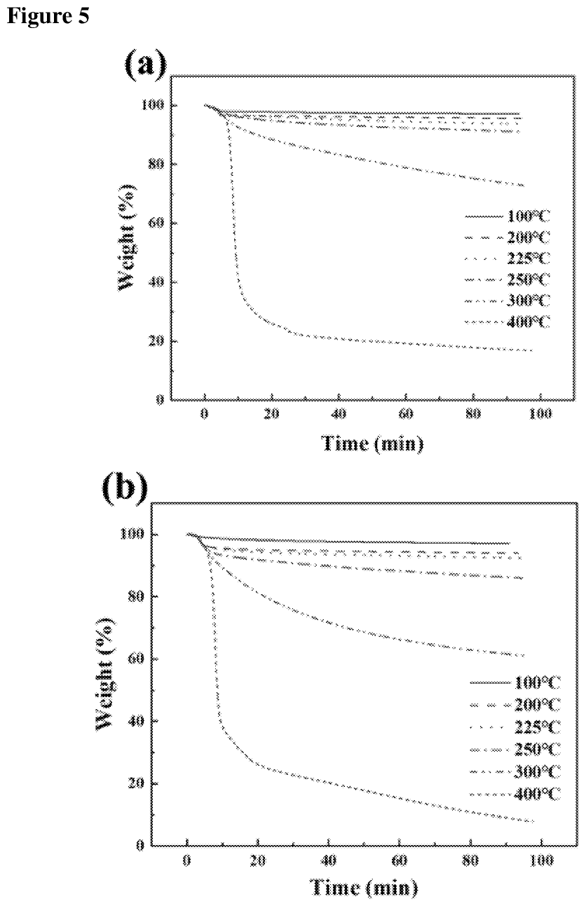

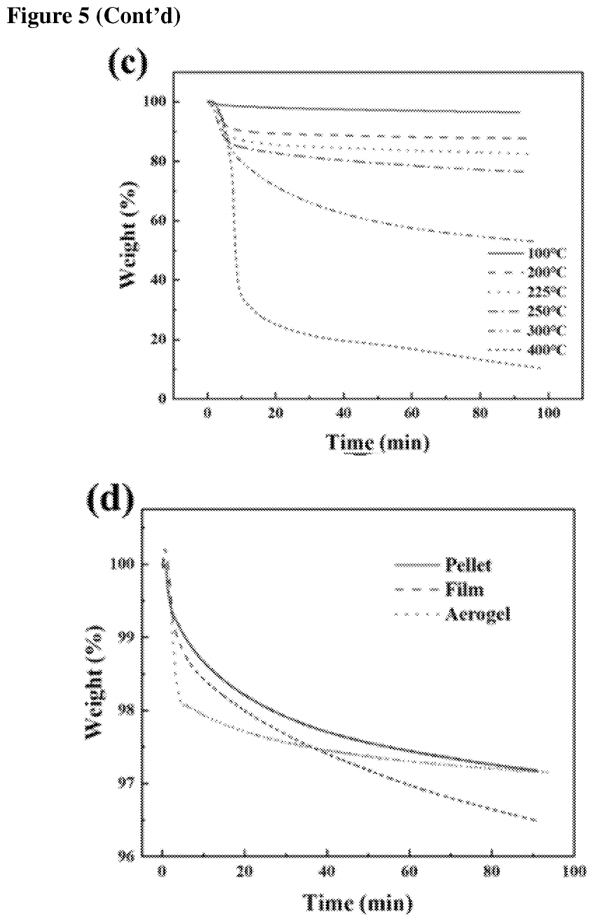

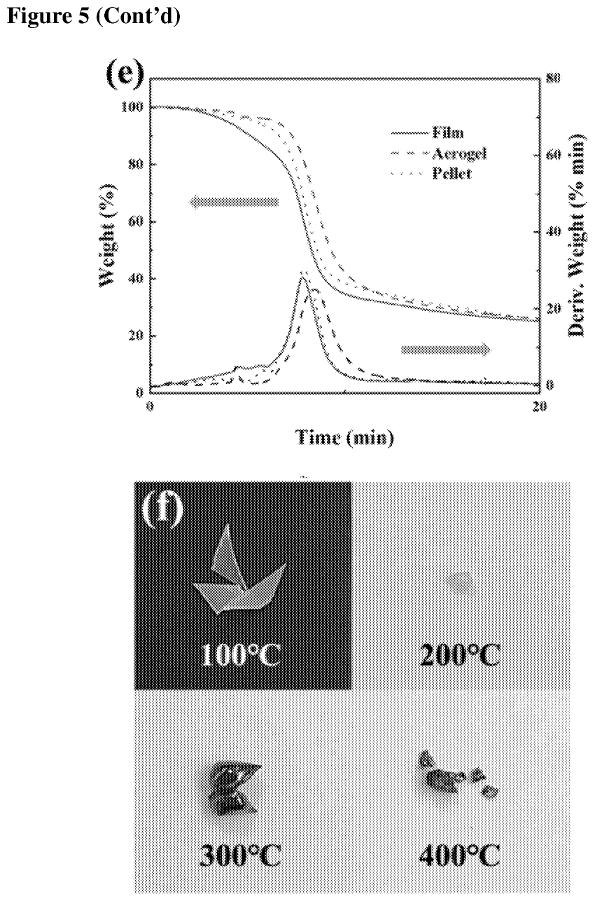

PVA pellets, PVA films (dried at ambient pressure), and one-step standing PVA aerogels (dried supercritically) were heated in a furnace under an air environment, maintaining the target temperature for a period to ensure that the weight loss occurred completely at that temperature. For each sample, thermal analysis was performed at different temperatures. As shown in FIG. 5, panels a-c, comparing the curve of weight percentage versus time of these three sample types at the same temperature reveals that aerogel has the highest remaining weight percentage. This means that the thermal performance of PVA aerogels is superior to both PVA pellets and PVA films. The comparison curve at 100° C. in FIG. 5, panel d shows that the aerogel's weight percentage decreases sharply in the initial stage. This is because, at lower temperatures, the initial weight loss is primarily due to the evaporation of absorbed moisture, and the porous structure of the aerogel facilitates the removal of moisture. Therefore, the weight loss decreases significantly and stabilizes at the lower temperature range. FIG. 5, panel e presents the weight loss curves and corresponding derivative curves for the three types of PVA products. It can be observed that the derivative curve of the PVA aerogel shifts to the right compared to those of the pellet and film, indicating that the aerogel exhibits superior thermal stability among the three samples. FIG. 5, panel f displays the residues of the PVA aerogel after heating at different temperatures. Based on the color, it is clear that heating at 100° C. did not cause the aerogel to lose its porous structure, as it still appears light blue. As the temperature increases, the aerogel gradually carbonizes. Table 1 shows the remaining weight percentage of the three samples after being held at different temperatures in a furnace for 90 min. These correspond to the lowest points of each curve in FIG. 5, panels a-c. The table demonstrates that at 200° C., 225° C., 250° C., 300° C., and 400° C., the remaining weight percentage of the PVA aerogel is the highest, indicating that the aerogel has better thermal stability.

TABLE 1
PVA aerogel, PVA pellet, and PVA film weight (%)
after 90 min at various temperatures.

| Samples | 100° C. | 200° C. | 225° C. | 250° C. | 300° C. | 400° C. |
|---|---|---|---|---|---|---|
| PVA aerogel | 97.15% | 95.68% | 93.80% | 91.11% | 72.79% | 16.77% |
| PVA pellet | 97.17% | 94.01% | 92.39% | 86.02% | 61.05% | 7.91% |
| PV film | 96.48% | 87.75% | 82.57% | 76.29% | 53.16% | 10.38% |

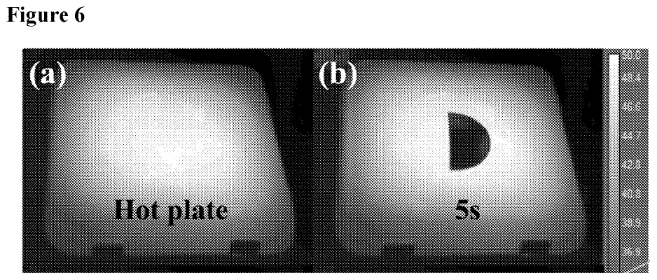

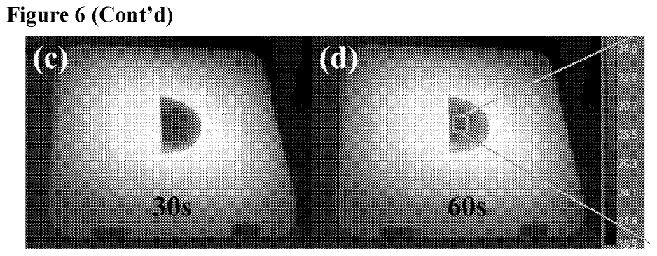

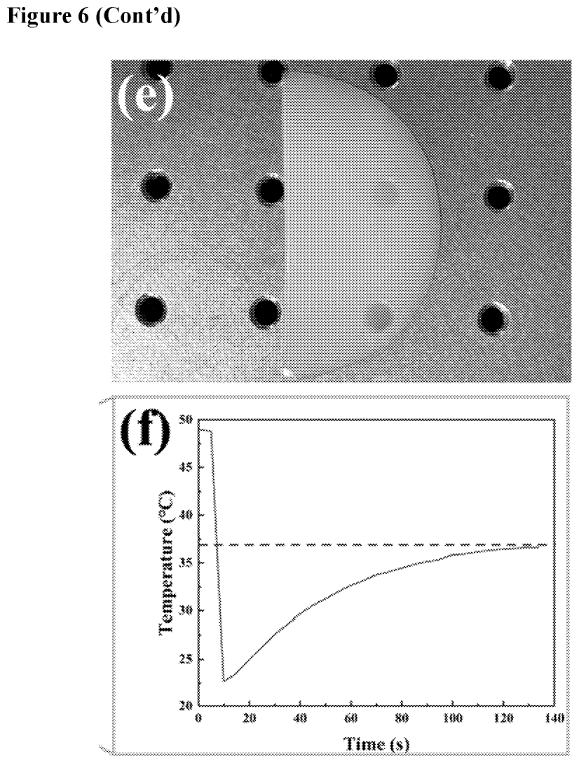

In exploring the thermal properties of PVA aerogels, direct heating tests were conducted, and a thermal camera was used to record the temperature of the aerogel. The direct heating test was conducted with 50° C. as the safe temperature. The sample was placed on a hot plate preheated to 50° C., and the process was recorded using a thermal camera. The image of the preheated hot plate under the thermal camera was captured and shown in FIG. 6, panel a, where it can be seen that the hot plate has reached the target temperature of 50° C. Subsequently, thermal images of the sample placed on the heated hot plate for 5 s, 30 s, and 60 s were shown in FIG. 6, panels b-d. It can be observed that, despite the sample being very thin, it still maintains a certain temperature difference with the hot plate, demonstrating the thermal insulation capability of the aerogel. FIG. 6, panel e shows the appearance of the sample after the test, which retained its transparency and light blue color, indicating that the internal porous structure was still intact. The curve shown in FIG. 6, panel f represents the temperature variation within a fixed area, which is marked by a blue box in FIG. 6, panel e. At the start of the curve, the temperature can be read as having already reached 50° C. After placing the aerogel at room temperature, a sharp drop in temperature to room temperature is observed. Over time, the temperature eventually stabilizes at 36.6° C., reaching a plateau while maintaining a temperature difference of over 10° C., providing a more intuitive demonstration of the aerogel's thermal insulation capability.

### CONCLUSIONS

Aerogels have recently attracted significant attention due to their unique properties, and a wide range of aerogels with various substrates have been developed. Among them, silica aerogel, the earliest and most established, stands out for its exceptional transparency and thermal insulation. However, its pearl necklace-like internal structure makes it highly brittle, severely limiting its practical applications. This challenge is addressed by developing an approach to enhance the mechanical properties of aerogels without compromising their transparency. Polyvinyl alcohol (PVA), a versatile polymer known for its self-cross-linking capabilities and long molecular chains, was chosen as the raw material. Unlike traditional aerogels, this study introduces a groundbreaking one-step standing method, a significant innovation in aerogel fabrication. This method drastically reduces the gelling time compared to the conventional freeze-thaw method and eliminates the need for external cooling equipment, making it both energy-efficient and eco-friendly. The result is a mechanically superior aerogel that retains high transparency, offering a promising new material for sustainable applications.

Several characterizations were performed afterward. Tensile testing using DMA revealed that the Young's modulus of the one-step standing PVA aerogel is 4.2596 MPa, significantly higher than that of silica aerogels, Cu NW aerogels, and graphene aerogels reported in the literature. SEM observations of carbon dioxide supercritical-dried PVA aerogel and ambient pressure-dried PVA film showed that the aerogel had a porous internal structure. A comparison between the one-step standing PVA aerogel and the freeze-thaw PVA aerogel revealed that the freeze-thaw aerogel had larger particle sizes in the hundreds of nanometers, consistent with Mie scattering. In contrast, the one-step standing aerogel exhibited a hierarchical porous structure, where the small-pore structure is nested within the skeletal framework of the large pores. The small pores measured tens of nanometers, consistent with Rayleigh scattering, which explains why it is transparent and exhibits a light blue color. This unique hierarchical pore structure opens up more possibilities for applications, such as drug delivery, where the large pores can store more drugs, and the small pores can ensure excellent mechanical performance while maintaining transparency. Further transmittance testing on the aerogels produced by the two types of gelling methods provided a more direct comparison of their effects on transparency. Since transparency is not only related to the material but also closely related to sample thickness, the parameter representing light transmission capability was calculated using Beer-Lambert law. The results indicated that the one-step standing PVA aerogel continued to exhibit superior light transmission, which is consistent with the SEM observations. Such a flexible and transparent material has potential applications in areas like solar energy conversion. Solar energy conversion devices often require materials that can allow sunlight to penetrate while also providing some thermal insulation to reduce energy loss. Aerogels have been considered for this photothermal application, but their brittle structure has limited their development. Therefore, the product described herein has a certain potential in the photothermal industry. Subsequent thermal performance tests were conducted using the method for recording weight loss during heating to examine PVA pellets, PVA aerogels, and PVA films. By comparing the weight loss curves at different temperature plateaus, the results showed that aerogel, indeed, had better thermal insulation properties. A comparison of the three curves at 100° C. revealed that the aerogel curve dropped more sharply at the beginning, further confirming the existence of its porous structure.

### Example 2: Effect of PVA Concentration on Aerogel Properties

### Further Materials and Methods

### Materials

Mowiol 56-98 PVA (Kuraray America Inc) was used as raw material. The molecular weight of it is about 105,600 g/mol, the degree of hydrolysis is 98.4±0.4 mol %, and the residual acetyl content is 1.5±0.4%. Dimethyl Sulfoxide (DMSO) (CAS #67-68-5, lot #472301) was bought from Sigma-Aldrich Corporation. Methanol. The series number of methanol is CAS #67-56-1, lot #A43420, purchased from Thermo Fisher Scientific. All the materials were used directly without any modification.

### Methods

Aerogels were formed through the sol-gel process. The term sol-gel process refers to solid nanoparticles dispersed in a liquid phase, which then aggregate and connect to form a three-dimensional framework that continues to extend within the liquid phase. Regarding the drying process, carbon dioxide supercritical drying was employed to ensure minimal structural collapse and maximize the porous structure's retention.

### Characterization

### Scanning Electron Microscope (SEM)

Scanning electron microscope (SEM) of model Zeiss Surpa 25 was used to observe the internal structure of PVA aerogels. This is a field emission microscope with high resolution. Scanning electron microscopy is a technique that uses a focused electron beam to scan the surface of a sample. The interaction of the electron beam with the atoms on the sample's surface allows for the visualization of the sample's morphology. Therefore, the sample's surface needs to be conductive, meaning that non-conductive polymer samples must be coated with a conductive metal layer. Thus, a Gatan high-resolution ion beam coater was used to apply a 15-20 nm thin layer of gold/palladium on the PVA aerogels. In the sample preparation process, liquid nitrogen is used to immerse the aerogel to make it brittle. Afterward, the aerogel is broken to obtain undamaged cross-sections, allowing for a more accurate observation of the aerogel's pore structure.

### Spectrophotometer (V-770)

The transparency of the aerogels was measured using a V-770 UV-visible/NIR spectrophotometer manufactured by JASCO Instrument, with a wavelength range from 190 nm to 2700 nm. The measurement range was set from 300 to 2500 nm, with a scanning speed of 200 nm/min and a UV/VIS Bandwidth of 5 nm. Using the same testing parameters, transmittance tests were conducted on PVA aerogels ranging from 10 to 15%.

### Dynamic Mechanical Analyzer (DMA)

Tensile tests were conducted using a dynamic mechanical analyzer (2710-103 series, manufactured by INSTRON Instrument). Tensile tests were performed on PVA aerogels at different concentrations (ranging from 10 to 15%) that underwent the same formation process. This was done to compare the impact of various concentrations on the mechanical properties of the aerogels.

### Results and Discussion

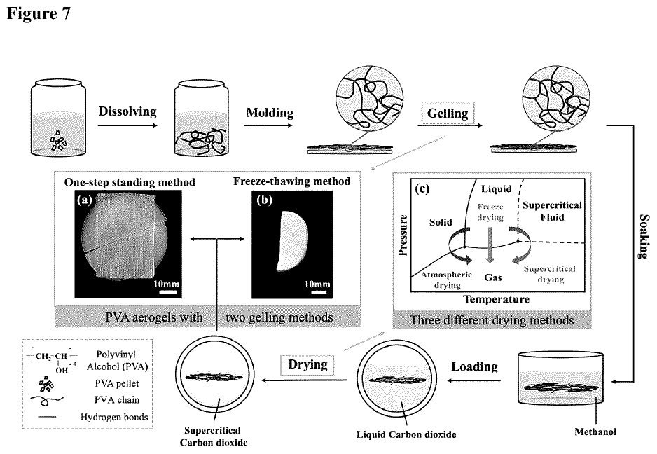

FIG. 7 shows the sample preparation process, starting with preparing the PVA solution. Next, the solution is dispensed into the molds, followed by the sol-gel process, immersion in the poor solvent for PVA to replace DMSO and demolding. Afterward, the wet gel is transferred to a supercritical dryer for carbon dioxide supercritical drying, resulting in PVA aerogels.

### Preparation Process

### Dissolving

An appropriate amount of polyvinyl alcohol (PVA) pellets was taken, DMSO was slowly added, and then stirred. The prepared PVA solution was transparent and viscous. The volume of DMSO and the corresponding mass of PVA required was calculated using Eq. (2),

$$\frac{x [ g ]}{x [ g ] + 1.1 [ \frac{g}{{cm}^{3}} ] V [ ml ]} = A \% \qquad ( 2 )$$

where x is the mass of PVA; V is the volume of DMSO; A % is the required concentration percentage of the PVA solution; 1.1 g/cm³ is the density of DMSO.

### Molding

The prepared PVA solution was transferred to the mold while hot. The choice of mold can control the final shape of the aerogel, which can be films, blocks, columns, and so on. The focus of the processes and aerogels described herein includes creating thin, uniform, and transparent aerogel sheets. As a result, after a series of mold selections, one type of particular mold was made to achieve this goal.

### Sol-Gel Process and Gelling

The classic sol-gel process was used. The prepared PVA solution was poured onto the preheated molds. With the help of the fluidity of the hot solution itself, the solution spread thinly and evenly on the mold. The mold was then placed in a sealed container and left to stand in a fume hood at room temperature to allow the long chains of PVA to cross-link and form a three-dimensional network structure. Hydroxyl groups, as side chains of polyvinyl alcohol, formed hydrogen bonds with each other to achieve physical cross-linking. The sample was then soaked in PVA-poor solvent (methanol), which replaced DMSO to obtain a wet gel. They were soaked for three days and replaced every 24 h with a fresh batch of pure methanol to speed up the replacement process.

Physical cross-linking was performed instead of introducing any chemical cross-linking agents. A standard method for physically cross-linking PVA hydrogels is the freeze-thawing method. As the name suggests, the freeze-thawing method involves repeated freezing and thawing cycles. During the freezing process, the crystallized state of the solvent limits the movement of the polymer chains, allowing adjacent hydroxyl groups enough time to form hydrogen bonds for physical cross-linking. Subsequently, the sample is thawed to restore the movement of the polymer chains, allowing more unpaired hydroxyl groups to come closer for a new round of freezing cross-linking. This repeated freeze-thawing cycle is used to maximize the formation of hydrogen bonds between molecular chains, thereby building a three-dimensional network. Since the freeze-thawing process generally requires three or more cycles, although the time and temperature of each cycle are consistent, it is not easy to ensure that the crosslinking points formed in different cycles are uniform and do not exhibit variations in density. In other words, the cross-linking process performed in different cycles results in significant differences in pore size within the three-dimensional network, limiting pore size uniformity within the aerogel. However, one essential factor related to aerogels' transparency is their internal structure's homogeneity. Therefore, a one-step crosslinking method was attempted to avoid the occurrence of non-uniform pore sizes due to the presence of solvent crystalline states, giving all hydroxyl groups an equal chance to form hydrogen bonds simultaneously.

The specific operation of the freeze-thawing cycles used was to transfer the prepared solution and the mold to a refrigerator for 24 h (at a temperature of approximately 4° C.). After freezing, the solution was no longer fluid and was removed and placed in a fume hood to thaw for 12 h (at a temperature of approximately 23° C.). This process was repeated for two cycles, and then a final freezing step is conducted. Finally, the frozen sample was directly immersed in methanol for replacement. In other words, the solution transferred into the mold underwent a freezing (4° C./24 h)-thawing (23° C./12 h)-freezing (4° C./24 h)-thawing (23° C./12 h)-freezing (4° C./24 h)-immersion in methanol cycle.

The one-step standing process involved placing the molded solution in a fume hood for 24 h for physical cross-linking and then directly immersing it in methanol. From an experimental procedure perspective, the one-step formation process was more straightforward and time-saving. Regarding the final aerogels, as shown in FIG. 7, panel a, a 12% aerogel was produced through the one-step standing method with high transparency. FIG. 7, panel b shows the 12% aerogel produced through the freeze-thawing method had very low transparency.

### Soaking

The purpose of the soaking process is to replace DMSO, which can dissolve PVA, with methanol, a poor solvent for PVA, as completely as possible. This aims to retain the three-dimensional network formed by the PVA skeleton only, known as the wet gel. The displacement process is based on the concentration difference of solvent between the inside and outside of the gel. Ideally, when pure methanol is replaced every 24 h, the longer the soaking time, the more complete the replacement, and the residual DMSO should reach 0%. However, in practice, it is impossible to remove DMSO altogether. Still, considering practical limitations and time efficiency during the experiment, the remaining DMSO should be kept as close to 0% as possible. Daily thermal gravimetric analysis (TGA) was conducted on PVA wet gels ranging from 10 to 15% to find the shortest soaking duration. The residual amount of DMSO was recorded daily while replacing an equal amount of methanol. The specific procedure of the thermal analysis was as follows: Ramp 5° C./min to 65° C., then isothermal at 65° C. for 10 min, followed by ramping at 5° C./min to 190° C. and an isothermal hold at 190° C. for 10 min. The selection of these two temperatures is based on the boiling point of methanol at 64.96° C. and the boiling point of DMSO at 189° C. The two isothermal temperatures were set slightly above the boiling points of methanol and DMSO, respectively. Isothermal for 10 min at each selected temperature allows both solvents to have enough time to evaporate. Then, the residual amount of DMSO was calculated and compared for each day to find the plateau. FIG. 8 shows that 10-15% of PVA gels reached a plateau in residual DMSO content on the third day. Therefore, in the daily replacement of methanol, the replacement time was set to three days.

### Drying

FIG. 7, panel c shows the path of three different drying methods in the phase diagram. Carbon dioxide supercritical drying was employed to ensure minimal structural collapse and maximize the porous structure's retention. Carbon dioxide has a critical temperature of 304.13 K (30.98° C.) and a critical pressure of 73.77 bar (1069.94 psi). The drying process involved transferring the wet gels after displacement (methanol displaces DMSO) to the supercritical dryer. Liquid carbon dioxide was then siphoned into the chamber of the dryer to soak the samples, displacing methanol with liquid carbon dioxide and leaving only liquid carbon dioxide in the gel pores. Every 24 h, all the liquid in the chamber was released, and new liquid carbon dioxide was siphoned in. Since carbon dioxide can maintain a liquid state only under high pressure, it turns into gas upon release. Therefore, all the liquid released from the chamber is methanol. Completion of displacement can be assessed by observing whether the liquid is still being released from the chamber. Based on this characteristic, it was determined that the displacement process takes two days. On the third day, there was no more liquid coming out. To ensure completeness, a final siphon of new liquid carbon dioxide was done after releasing it on the third day, and then the drying process could be immediately initiated.

An E3100 critical point dryer from Electron Microscopy Sciences company was used. The chamber of the dryer is designed with a double-layer structure. The inner layer is the chamber where the samples are placed. The outer layer has inlets and outlets for circulating hot water to heat the inner chamber. Heating the outer layer with circulating hot water can increase the temperature and pressure in the inner chamber and make the carbon dioxide reach a supercritical state. The dryer was equipped with temperature and pressure gauges and a vent valve to control the internal pressure. Typically, the pressure is regulated at 1200 psi, and the temperature was increased to 35° C. The chamber was maintained for 90 min to ensure all carbon dioxide is supercritical. After that, a slow depressurization ensures the carbon dioxide returned to the gaseous state, avoiding the state transition process across the boundary between liquid and gas. This helped prevent structural collapse due to surface tension.

### Challenges

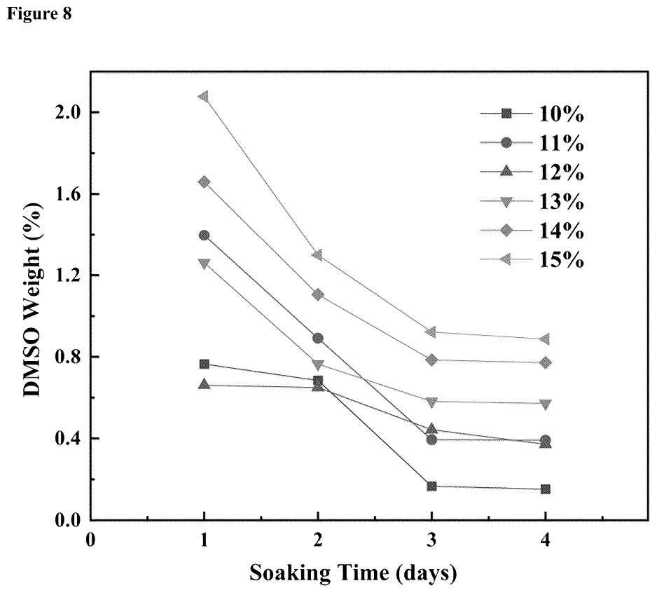

During the development process of aerogel formulations, some challenges were encountered. Experiments were conducted on aerogels with concentrations of PVA ranging from 2 to 28%. Initially, the challenge faced was that the finished aerogels were uneven and curled after drying, as shown in FIG. 9, panels a, b, and c. When the concentration was too low (below 10%), the concentration of PVA chains in the solution was insufficient to form a stable three-dimensional network structure. It was unable to maintain a sheet-like morphology when immersed in methanol but dispersed into floccules or small pieces. FIG. 9, panels d, e, and f show that increasing the concentration of the aerogel can partially resolve the issue of shape, gradually leading to smoother, sheet-like samples. Then, attempts were made to further address this issue using the freeze-thawing method, as samples in a crystalline state after freezing would avoid immediate dispersion upon immersion in methanol. However, during the high-temperature and high-pressure drying process, the problem of not maintaining a flat sheet-like form still occurred due to the instability of the skeleton structure.

Gradually, the PVA concentration was increased, and minor improvements were made to the experimental process, gradually achieving flat aerogels. To further obtain aerogels with higher transparency, the formulation was further improved. A one-step gelation method was adopted to obtain aerogels with higher transparency. However, new challenges were encountered; when the concentration was too high (above 15%), the viscosity of the solution became too high. This led to the formation of many bubbles during the dissolution and pouring of the solution, resulting in non-uniform bubbles within the sample, significantly affecting the transparency of the aerogel, as shown in FIG. 9, panels g, h, and i. Therefore, it was finally decided to control the PVA concentration within the safe range of 10-15% to ensure continuous stable production of aerogels with smooth, uniform, and highly transparent morphology, as shown in FIG. 9, panels j, k, and l.

### Properties

### Mechanical Properties

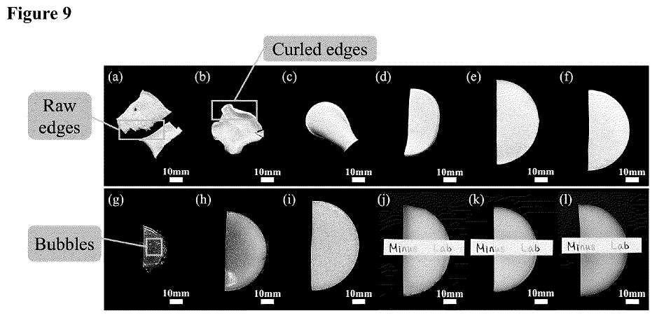

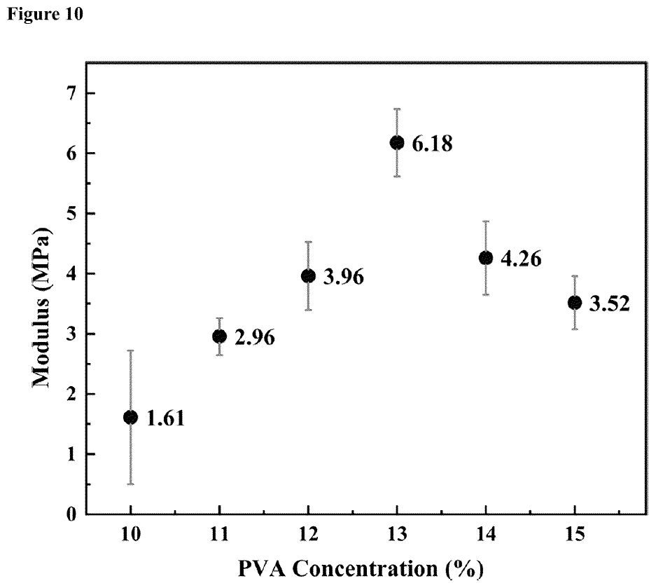

With the increase in PVA concentration in the sample, the number of free hydroxyl groups also increases. Although an increase in hydroxyl groups can facilitate the formation of hydrogen bonds to establish a three-dimensional network structure, there are many factors that influence the formation of this network, including the crystallinity of the sample, the number of hydrogen bonds, and the uniformity of their distribution. Hydroxyl groups are side groups on the long chains of PVA, and their ability to come close enough to form hydrogen bonds is also related to the rigidity of the PVA chains. Therefore, the concentration and mechanical properties cannot be theoretically assumed to have a simple linear relationship. The effect of concentration on mechanical performance is summarized through experiments. Tensile tests were conducted using a dynamic mechanical analyzer (2710-103 series, manufactured by INSTRON Instrument). Tensile tests were performed on PVA aerogels at different concentrations (ranging from 10 to 15%) that underwent the same formation process. This was done to compare the impact of various concentrations on the mechanical properties of the aerogels. From FIG. 10, the sample with a 13% concentration has the highest Young's modulus, reaching 6.18±0.56 MPa. The Young's modulus of regular PVA film is 2.32±0.3 MPa. Based on the data from the graph, it is evident that PVA aerogels, despite having a porous structure, can still maintain the excellent mechanical properties of PVA. In some cases, they even exhibit a higher Young's modulus.

### Morphology

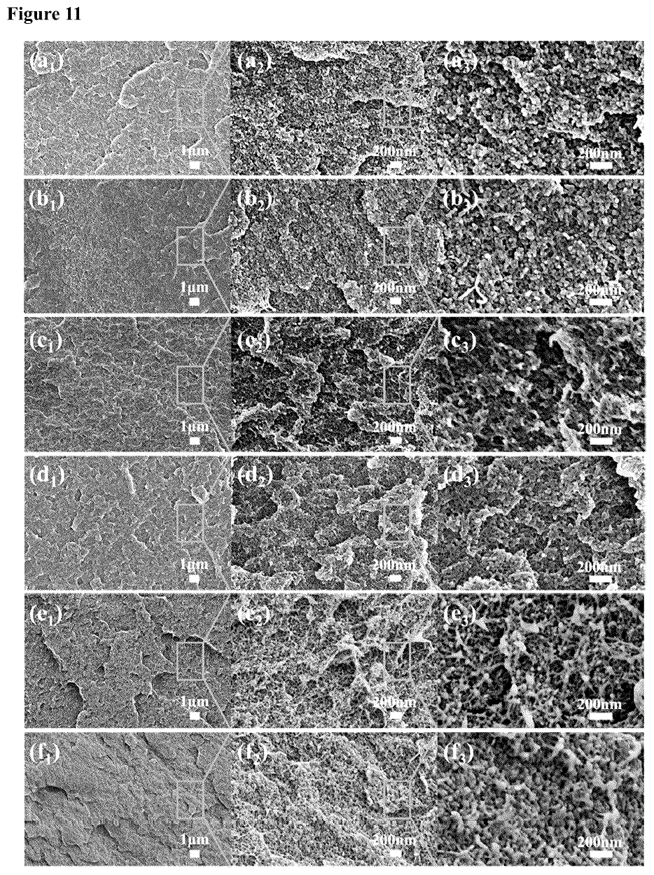

SEM tests were conducted on 10-15% of PVA aerogels to compare their internal structures. To easily observe their internal structures, it was necessary to expose the cross-sections of the aerogels. PVA aerogels are very flexible and resilient, making them difficult to break directly. If cut with a knife, the cutting process exerts external pressure on the aerogels, leading to the collapse of the three-dimensional network. Liquid nitrogen (at a temperature of approximately −195° C.) was used to make the aerogel brittle, allowing for easy breaking and obtaining relatively intact cross-sections. As mentioned earlier, the sample surface for SEM observation needs to be conductive, so a sputter coater was used to coat the samples. The sputter coater could sputter 2 nm platinum (Pt) grains onto the sample surface. FIG. 11 shows SEM images of 10-15% aerogels at different magnifications. When observed horizontally in the images, it is easy to compare the internal structure morphology at the same concentration but different magnifications. When observed vertically, it allows for comparing the internal structures of aerogels at the same magnification but at different concentrations. Vertical observation in FIG. 11 shows that the internal pore distribution of 10% and 11% PVA aerogels is relatively uniform. Starting from 12%, it is observed that some PVA chains begin to crowd together, and this crowding continues until the 14% PVA aerogel, where crowded PVA clusters are still present. The proximity of polymer chains results in a gradation of pore sizes. This means that the pores between PVA clusters are more prominent, approximately in the range of a few hundred nanometers. When the magnification is further increased, numerous secondary pores are found within each large pore, and their sizes are on the same order of magnitude as the pores in the 10% and 11% PVA aerogels. Further observing of the SEM image of the 15% PVA aerogel reveals that the pore gradation has been mitigated, and the distribution of PVA chains appears more uniform.

FIG. 11, panels (a) to (f) represent PVA aerogels of different concentrations from 10 to 15%, while subpanels (1) to (3) represent magnifications of 5000×, 30,000×, and 60,000×, respectively.

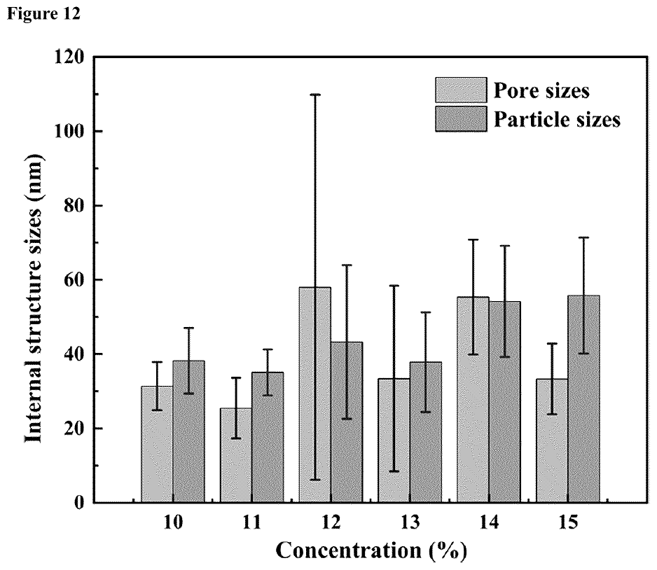

The pore size and particle size data were obtained by analyzing SEM images using ImageJ, with each data point based on an average of 50 measurements. The standard deviation was also recorded. The size of the standard deviation corresponds to the uniformity of the pores. From FIG. 12, the samples are ranked from lowest to highest standard deviation as follows: 10%, 11%, 15%, 14%, 13%, and 12%. This means that the 10% sample has the best pore uniformity, while the 12% sample has the worst. Similarly, for PVA aerogels with concentrations ranging from 10 to 15%, their pore sizes are of the same order of magnitude. Regarding light scattering, there are two classical definitions: Rayleigh Scattering and Mie Scattering. Rayleigh Scattering refers to the scattering that occurs when the particle size is much smaller than the wavelength, typically explaining everyday phenomena such as why the sky is blue and why it turns red at sunset. Mie Scattering, on the other hand, refers to scattering when the particle size is equal to or larger than the wavelength, which often explains the phenomenon of brownish smog. By considering the skeleton width in SEM images of PVA aerogels as the particle diameter, and performing at least 50 measurements, the particle size within the internal structure of samples with concentrations from 10 to 15% can be determined. The particle sizes within PVA aerogels at different concentrations are quite different compared to the wavelength, leading to Rayleigh Scattering when light interacts with the material. This also explains why PVA aerogels exhibit a light blue color.

### Transmittance

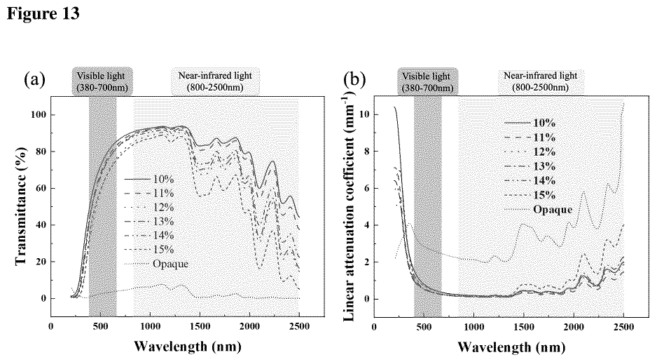

Transmittance tests were conducted on PVA aerogels ranging from 10 to 15%. The tests were performed using a V-770 UV-Visible/NIR spectrophotometer with a measurement range set from 300 to 2500 nm, a scanning speed of 200 nm/min, and a UV/VIS bandwidth of 5 nm. FIG. 13, panel a shows the curves comparing transmittance with wavelength for PVA aerogels of different concentrations. The 10% sample exhibited higher transmittance. The wavelength range within the blue box in FIG. 13 represents the visible light range, while the red box indicates the near-infrared light range. At a wavelength of 1333 nm, the transmittance reaches its highest value of 93.67%.

Transmittance is influenced not only by the sample's internal structure but also by the thickness. Since the directly tested samples had slightly varying thicknesses, the thickness effect on transmittance was accounted for using Beer-Lambert's law (Eq. 1), where Tis transmittance [%]; x is the thickness of aerogel [mm]; a is linear attenuation coefficient [mm⁻¹]. The linear attenuation coefficient refers to the difficulty of propagation of specific physical quantities, such as photons, sound waves, and electrons, in a material along a single direction. For assessing the transparency of materials, a higher linear attenuation coefficient of the material itself implies that light entering the material will rapidly attenuate. In contrast, a lower linear attenuation coefficient suggests light can more easily pass through the material. In other words, the linear attenuation coefficient for different concentrations of PVA aerogel is calculated by eliminating the effect of the PVA aerogel's thickness. A lower coefficient represents a more transparent material.

From FIG. 13, panel b, it can be observed that the aerogels prepared by the one-step standing method exhibit excellent light-transmitting ability, regardless of concentration, which is far superior to the opaque aerogels produced by the freeze-thawing method. In the 700 nm to 1300 nm range, the effect of PVA concentration on the aerogel can be ignored, as all one-step standing aerogels exhibit excellent light-transmitting ability. As the wavelength continues to increase, it can be observed that the 11% sample has better light-transmitting ability, while the 15% sample has poorer light-transmitting ability. This can provide some guidance for the application of such aerogels in different fields, helping to balance cost, transparency, and even mechanical and thermal insulation properties to choose the most suitable formulation.

### CONCLUSIONS

At first, considering the brittleness and poor mechanical properties of the earliest discovered silica aerogels, this drawback limits the application development of aerogels. Therefore, improving the mechanical properties of aerogels was set as the primary goal. Thus, the material selection focused on polymers. The experimental direction was set as pure physical cross-linking without using any cross-linking agents to simplify and expedite the production process.

Thus, polyvinyl alcohol (PVA), which can undergo self-crosslinking through side-chain hydroxyl groups forming hydrogen bonds, was chosen as the raw material to attempt aerogel production. Mechanical tests were conducted on transparent PVA aerogels with concentrations ranging from 10 to 15%. The sample with a 13% concentration achieved the highest modulus, reaching 6.18±0.56 MPa.

After the material selection, the production method was experimented with, analyzed, and refined. Comparative analysis of freeze-thawing methods revealed that this approach could not guarantee the transparency of PVA aerogels. Considering the time efficiency of the experimental process, a one-step standing method was invented to facilitate the sol-gel transition. The one-step standing method not only maintained high transparency of PVA aerogels but also reduced the time required for the gelation process compared to the freeze-thawing method. Subsequently, PVA concentrations ranging from 2 to 28% were tested, revealing various challenges, including curling, raw edges at low concentrations, and bubble formation at high concentrations. The production process was further refined, and a safe concentration window of 10-15% was determined, ensuring the production of stable, thin, and transparent PVA aerogel.

Using ImageJ, SEM images of samples with concentrations ranging from 10 to 15% were analyzed, with no fewer than 50 measurements taken for each sample to determine pore sizes. The standard deviation of pore sizes was recorded, as it can represent the uniformity of pore sizes. Pore sizes are within the same order of magnitude for samples with 10% and 15% concentrations, mostly in the tens of nanometers. Additionally, the internal skeleton diameter can be considered the particle size for open-pore structured aerogels. The particle size of PVA aerogels described herein is much smaller than the wavelength, satisfying the conditions for Rayleigh Scattering, which explains why the aerogels appear light blue. The light transmittance curve and linear attenuation coefficient versus the wavelength curve of the opaque samples were also compared with those of the transparent samples. The opaque samples prepared by the freeze-thawing method have significantly lower transparency than those prepared using the improved one-step standing method described herein.

### INCORPORATION BY REFERENCE

All U.S. and PCT patent application publications and U.S. patents cited herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. An aerogel comprising polyvinyl alcohol (PVA);
wherein the aerogel comprises a plurality of small pores having an average small pore diameter; a plurality of large pores having an average large pore diameter; and
the average small pore diameter is less than the average large pore diameter.

2. The aerogel of claim 1, wherein the aerogel has a Young's modulus of about 0.01 MPa to about 6 MPa.

3. The aerogel of claim 1, wherein the aerogel has a Young's modulus of about 1 MPa, about 2 MPa, about 3 MPa, about 4 MPa, about 5 MPa, or about 6 MPa.

4-8. (canceled)

9. The aerogel of claim 1, wherein the plurality of small pores and the plurality of large pores are substantially uniformly distributed in the aerogel.

10. The aerogel of claim 1, wherein the average small pore diameter is about 10 to about 100 nm.

11. The aerogel of claim 1, wherein the average large pore diameter is about 100 nm to about 1000 nm.

12. The aerogel of claim 1, wherein the aerogel comprises a plurality of particles having a particle size of about 10 to about 100 nm.

13. The aerogel of claim 1, wherein the aerogel exhibits Rayleigh scattering.

14. The aerogel of claim 1, wherein the aerogel has transmittance values of about 30% to about 85% in the visible light spectrum (380-700 nm).

15. The aerogel of claim 1, wherein the aerogel has transmittance values of about 10% to about 95% in the near-infrared light spectrum (800-2500 nm).

16. The aerogel of claim 1, wherein the aerogel has transmittance values of about 90% to about 94% in the near-infrared light spectrum (800-2500 nm).

17. The aerogel of claim 1, wherein a portion of the small pores are positioned within the large pores.

18. A building material, comprising the aerogel of claim 1.

19. The building material of claim 18, wherein the building material is selected from windows and facades.

20. A medical device, comprising the aerogel of claim 1.

21. The medical device of claim 20, wherein the medical device is a dressing.

22. Aerospace equipment, comprising the aerogel of claim 1.

23. A solar panel cover, comprising the aerogel of claim 1.

24. A filter, comprising the aerogel of claim 1.

25. A process of preparing an aerogel comprising polyvinyl alcohol (PVA), comprising:
combining PVA and a first solvent, thereby producing a PVA solution;
introducing the PVA solution into a mold;
allowing the PVA solution to gel at ambient temperature for a gelling duration, thereby producing a PVA wet gel;
contacting the PVA wet gel with a second solvent to form a soaked PVA wet gel; and
drying the soaked PVA wet gel, thereby producing a PVA aerogel.

26.-55. (canceled)
